(19) United States
(12) Patent Application Publication
(10) Pub. No.: US 2005/0129789 A1
(43) Pub. Date: Jun. 16, 2005

(54) Drugs including anticancer agents and immunopotentiators and health foods and drinks

(76) Inventor: Yasuyuki Shirota, Aomori (JP)

Correspondence Address: WITHROW & TERRANOVA, P.L.L.C., P.O. BOX 1287, CARY, NC, 27512, US

(21) Appl. No.: 10/509,642

(22) PCT Filed: Mar. 28, 2003
(86) PCT No.: PCT/JP03/03944

(30) Foreign Application Priority Data
Mar. 29, 2002 (JP) 2002-95590

(51) Int. Cl. A61K 35/78

(52) U.S. Cl. 424/765

(57) ABSTRACT

It has been proven that a fruit of the genus Malus such as apple (Malus domestica) and crabapple (Malus pumila), a fruit of the genus Pyrus, or a substance derived therefrom, when administered, has the actions of enhancing innate immune system such as natural killer cells, macrophages and the like, activity of lymphocytes, to thus control cancer-attacked cells, as well as to prevent carcinogenesis of cells not yet affected by cancer, and when taken regularly, further has an effect of preventing the onset of cancer, i.e., having a cancer-preventing effect. It has been further proven that both a mature apple and an immature apple, or an immature apple alone has a similar action of preventing the onset of cancer.

### TECHNICAL FIELD

The present invention relates to drugs and health foods including anticancer agents and immunopotentiators for inhibiting and preventing cancer and for enhancing immune system, comprising a fruit of the genus Malus such as apple (Malus domestica) and crabapple (Malus pumila), a fruit of the genus Pyrus such as a pear, or a substance derived therefrom as a raw material.

Specifically, the present invention relates to drugs and health foods including anticancer agents and immunopotentiators for inhibiting and preventing cancer and for enhancing immune system, comprising as a raw material the above-mentioned fruit of the genus Malus, the fruit of the genus Pyrus, a substance derived therefrom as it is or as processed to juice and the like, an extract obtained by extracting the above-mentioned fruit of the genus Malus or the fruit of the genus Pyrus with water or an organic solvent such as alcohol, or a substance derived from the extract. The present invention further relates to drugs and health foods including anticancer agents and immunopotentiators for inhibiting and preventing cancer and for enhancing immune system, comprising a mature fruit and/or an immature fruit of the genus Malus such as apple (Malus domestica) and crabapple (Malus pumila), or a fruit of the genus Pyrus as a raw material.

### BACKGROUND ART

Conventionally, it has been known that an extract of Cordyceps sinensis, when administered as a drug such as an anticancer agent or an immunopotentiator and a health food, has the effects of enhancing the innate immune system such as natural killer cells, macrophages and the like, in addition to suppressing the growth of existing cancer cells, and when taken regularly, it further has the effect of preventing the onset of cancer, that is, the cancer preventing effect (for example, see Patent Document 1).

In addition, it has also been known that as a health food containing apple juice, which is prepared by mixing apple juice and a proantocyanidine-containing substance, powdering the mixed solution to prepare a powdered composition to subsequently obtain a health food containing the powdered composition of apple juice as an active ingredient, has anti-oxidation activity, anti-mutagenic activity, anti-ulcer activity, deodorization effect for excrement and preventing effect for diabetic complications, due to the presence of proantocyanidine, in addition to the effects of the natural ingredients derived from the fruit juice itself (for example, see Patent Document 2).

[Patent Document 1]

JP-A-1989-228440 (page 1, page 4, FIG. 1).

[Patent Document 2]

JP-A-2001-275604 (page 1, page 7).

The extract of Cordyceps sinensis has been conventionally used in such a drug and a health food. However, since Cordyceps sinensis is expensive and is not easily available, it has problems in that it cannot be widely administered.

In addition, there has been no mention that conventionally known health foods containing apple juice have therapeutic effects of an anticancer agent, an immunopotentiator or the like.

On the other hand, apple Malus domestica is a fruit tree which belongs to the family Rose having a long history, which is presumably indigenous to the region from West Asia to Middle-east Asia, having been cultivated for 4000 years.

Apple Malus domestica which is currently cultivated in Japan may be one which was imported in the Meiji dynasty or one which has been improved through breeding since then. In Europe, it has been known that the pectin of the plant fiber contained in the fruit has the effects of establishing a healthy balance in the intestine as said in the quotation “an apple a day keeps the doctor away”. Further, apple has been known to contain a large amount of potassium which has a diuretic action and fructose (glucose) which is a source of energy.

However, medical or pharmaceutical studies on the pharmacological effects of the apple have been rarely carried out, but the similar studies on health food containing apple vinegar as a supplementary ingredient and immune system strengthening food containing apple (Malus domestica) have been proposed. However, studies on the anticancer action or the immunopotentiating action of the apple have not been conducted, except for the research by Miura et al., School of Medicine at Hirosaki University, which reports the finding that apple polyphenol has an antitumor effect.

In other words, a specific study related to the pharmacological effects of apple itself has not been carried out, and the mechanism of the pharmacological effects has not yet been clarified, and further studies on apple as an anticancer agent, an immunopotentiator, or a cancer-preventing agent have not been tried at all.

Currently, the leading cause of death in Japan is cancer, and surgical operation, radioactive treatment, chemotherapy using an anticancer agent, etc. are commonly carried out as treatments for cancer. However, cancer for which anticancer agents are effective is limited, and further the side effects from the anticancer agents including death are becoming socially important problems.

On the other hand, humans and animals have inherent immune system which protects the body from pathogenic microorganisms. Since cancer cells are self-derived foreign substances generated in the body, in the case where the immunity is high, the cancer cells are typically removed by natural killer cells, macrophages and the like.

However, if the immunity is lowered due to aging or accumulated stress, cancer cells tend to proliferate.

An object of the invention described in claims 1 to 3 and 6 in the present inventions is to enhance the innate immune system such as natural killer cells, macrophages and the like, or the activity of lymphocytes, and thus prevent or suppress cancer in addition to inhibiting the growth of cancer cells by using a fruit of the genus Malus such as apple (Malus domestica) and crabapple (Malus pumila), or a fruit of the genus Pyrus.

An object of the invention described in claim 4 is to enable suitable administration depending on the situation, in addition to the object of the invention described in claims 1 to 3.

An object of the invention described in claims 5 and 7 is to obtain a higher antitumor effect or an immunopotentiating effect, in addition to the object of the invention described in claims 1 to 4 or 6.

### DISCLOSURE OF INVENTION

The present inventors have found that a fruit of the genus Malus such as apple (Malus domestica) and crabapple (Malus pumila), a fruit of the genus Pyrus, or a substance derived therefrom, has an anticancer effect or a cancer-suppressing effect by initially carrying out various animal tests, and then on humans, to thus complete the present inventions.

To achieve the above-mentioned objects, the invention described in claim 1 in the present inventions is directed to a drug such as an anticancer agent or an immunopotentiator, which is characterized by comprising a fruit of the genus Malus such as apple (Malus domestica) and crabapple (Malus pumila), a fruit of the genus Pyrus, or a substance derived therefrom as an active ingredient.

The invention described in claim 2 is characterized by the constitution of using an aqueous extract of the fruit of the genus Malus such as apple (Malus domestica) and crabapple (Malus pumila), the fruit of the genus Pyrus, or a substance derived from the aqueous extract, in addition to the constitution of the invention described in claim 1.

The invention described in claim 3 is characterized by the constitution of using an extract of the fruit of the genus Malus such as apple (Malus domestica) and crabapple (Malus pumila), the fruit of the genus Pyrus obtained by extraction with an organic solvent such as alcohol, or a substance derived from the extract, in addition to the constitution of the invention described in claim 1.

The organic solvent such as alcohol herein includes, for example, acetone, ether, ethyl acetate, chloroform, and methylene chloride.

It has been revealed that the effects of invention described in claims 1 to 3 obtained from such constitutions, consist in that a fruit of the genus Malus such as apple (Malus domestica) and crabapple (Malus pumila), a fruit of the genus Pyrus, a substance therefrom, an extract obtained by extracting the fruit with water or an organic solvent such as alcohol, or a substance derived from the extract, when administered, enhances the immune system such as natural killer cells, macrophages and the like to control cancer-attacked cells, as well as to prevent carcinogenesis of cells not yet attacked by cancer, and when taken regularly, has the effect of preventing the onset of cancer, that is, the cancer preventing effect.

The invention described in claim 4 is characterized by the constitution of administering as a raw material an extract of the fruit of the genus Malus such as apple (Malus domestica) and crabapple (Malus pumila), the fruit of the genus Pyrus, or a substance derived from the extract orally, percutaneously, parenterally, or via other routes, in addition to the constitution of the invention described in claim 1, 2 or 3.

The effect of invention described in claim 4 obtained from such constitution in addition to the effects of the invention described in claim 1, 2 or 3, consists in that it enables percutaneous administration, parenteral administration and administration via other routes, although oral administration is a general administration route.

The invention described in claim 5 is characterized by the constitution of comprising as a raw material either or both of a mature fruit and an immature fruit of the genus Malus such as apple (Malus domestica) and crabapple (Malus pumila), or the fruit of the genus Pyrus, in addition to the constitutions of the invention described in claim 1, 2, 3 or 4.

It has been revealed that the effect of invention described in claim 5 obtained from such constitution in addition to the effects of the invention described in claim 1, 2, 3 or 4, consists in that a mixture of a mature apple and an immature apple, or an immature apple alone has a similar action of preventing the onset of cancer.

The invention described in claim 6 is a health food for inhibiting and preventing cancer or for enhancing immune system, comprising a fruit of the genus Malus such as apple (Malus domestica) and crabapple (Malus pumila), a fruit of the genus Pyrus, or a substance derived therefrom as a raw material.

It has been proved that the effect of invention described in claim 6 obtained from such constitution consists in that a fruit of the genus Malus such as apple (Malus domestica) and crabapple (Malus pumila), a fruit of the genus Pyrus, or a substance derived therefrom, when administered, enhances the immune system such as natural killer cells, macrophages and the like to control cancer-attacked cells, as well as to prevent carcinogenesis of cells not yet attacked by cancer, and when taken regularly, has the effect of preventing the onset of cancer, that is, the cancer preventing effect.

The invention described in claim 7 is characterized by the constitution of comprising either or both of a mature fruit and an immature fruit of the genus Malus such as apple (Malus domestica) and crabapple (Malus pumila), or the fruit of the genus Pyrus as a raw material, in addition to the constitution of the invention described in claim 6.

It has been revealed that the effect of invention described in claim 7 obtained from such constitution in addition to the effect of the invention described in claim 6, consists in that a mixture of a mature apple and an immature apple, or an immature apple alone, has a similar action of inhibiting the onset of cancer.

### BRIEF DESCRIPTION OF THE DRAWINGS

FIG. 1 is a graph showing a tumor growth curve (mean value) of mice from each test group after tumor MethA inoculation.

FIG. 2 is a graph showing the phagocytosis ability of peritoneal macrophages of mice having no tumor inoculation and in mice in which tumor has been cured after tumor inoculation.

FIG. 3 is a graph showing the change of the activity of natural killer cells in a subject orally administered with apple in a form of juice every day for 5 weeks.

### BEST MODE FOR CARRYING OUT THE INVENTION

In the following, Example of the present invention will be explained with reference to Drawings.

This Example shows the case where fruit juice of apple Malus domestica was orally administered as a fruit of the genus Malus.

40 female 4-week-old BALB/c mice (CLEA Japan, Inc.) were divided into four test groups (10 mice/group), i.e., a test group administered with fruit juice of apple Malus domestics, a group administered with the extract of the fruiting body of Cordyceps sinensis, a test group administered with a mixture of the extract of the insect body part of Cordyceps sinensis, and a control group administered with water alone.

100 grams of Cordyceps sinensis were decocted with 1 liter of distilled water for 70 minutes to prepare a stock solution of the extract of Cordyceps sinensis.

The fruit juice of apple Malus domestics was prepared by a juicer using a water-washed fruit with the fruit skin (containing a large amount of polyphenol or quercetin) included and the fruit core (including a seed) removed, and was then used without heat-treatment or antioxidant treatment.

Both of the extract of Cordyceps sinensis and the apple juice were frozen and kept at −35° C., thawed out every day, and mixed with edible water.

Tap water sterilized with an autoclave was used as the edible water, which was changed every day The apple juice or the extract of Cordyceps sinensis was incorporated into the edible water, and the mice were allowed free access to water. Each of the concentrations was 2% by volume.

The mice were allowed free access to food which was solid feed for mice (CLEA Japan, Inc.), and the amount of water taken by the mice was recorded as well as the amount of the food taken.

Using a big cage, 5 mice per cage were raised.

All the mice were identified individually, and each of the body weights was recorded every day.

The inside of the cages was kept at a temperature of 25° C. and a humidity of 55%, and the illumination was set such that light:dark=12 hours:12 hours.

The mice were allowed to live freely from 4-week-old to 9-week-old, and in the middle of ninth week, the mice were inoculated with 1×10⁵ of mouse tumor MethA by subcutaneous injection.

The hairs of the abdominal side of the inoculation site were shaved before inoculation to carry out a precise observation for establishment of the tumor. The short diameter and the long diameter of the tumor were measured with a digital caliper every day, which was used as a reference for determining the anticancer effect.

All of the mice which died from the cancer were dissected and investigated for the metastasis of tumor to other organs and for the existence of ascites, and were kept in a formalin solution, and suitable histological analysis was also carried out.

Life time was significantly prolonged and therefore it was evident that each of the mice from the group administered with the fruit juice of apple (Malus domestica) had benefited from the antitumor effect, as compared to the control group administered with water alone after the inoculation of tumor MethA.

Further, the size of tumor diameter was remarkably reduced as compared to the control group or the other test groups, which clearly indicated the antitumor effect of apple.

In the following, the detailed description will be provided.

One mouse in the control group administered with water alone died first on the 25^{th} day after the inoculation of tumor MethA, and then one mouse in the group administered with Cordyceps sinensis died on the 28^{th} day. However, no death was observed from that day until the 32^{nd} day.

Further, no death was observed in the group administered with the apple juice until the 35^{th} day. The day for 50% death was the 44^{th} day for the Cordyceps sinensis group and the 53^{rd} day for the group administered with apple juice as compared to the 39^{th} day for the control group, which indicates the life-prolonging effects by 5 days and 14 days, respectively as compared to the control group.

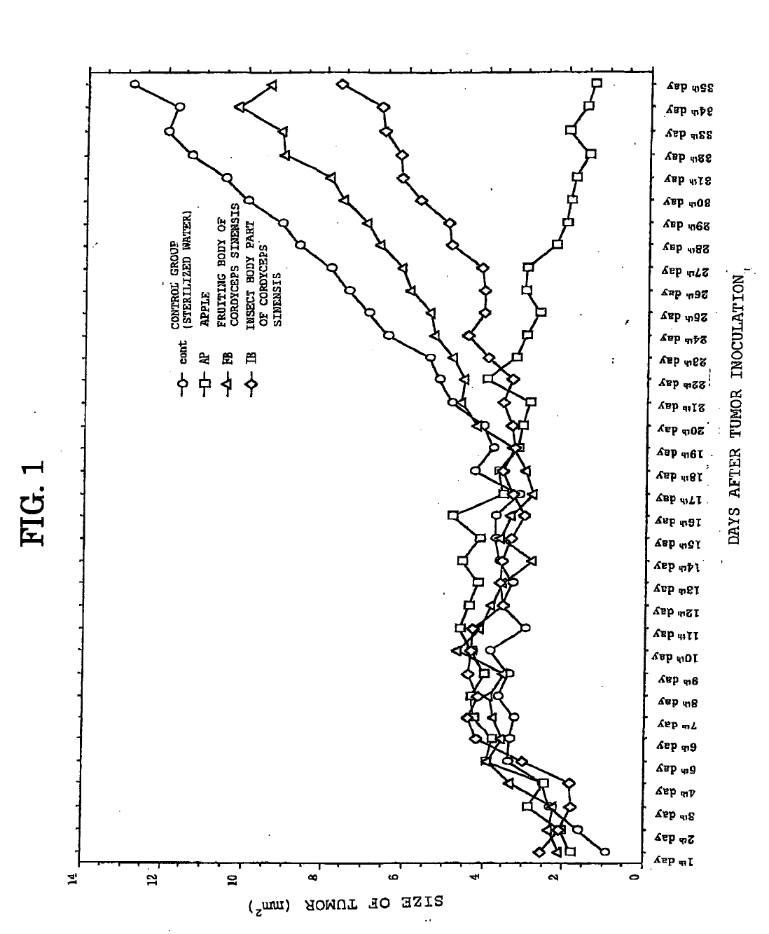

Next, the tumor size change of subject mice in each test group until the 35^{th} day after inoculation of tumor MethA was shown in FIG. 1.

As the results, as shown in FIG. 1, the tumor growth rate of mice in the group administered with apple juice was suppressed to about ¼ as compared to the Cordyceps sinensis group, and about 1/10 as compared to the control group administered with water alone.

As the results of Fisher's PLSD assay, these values statistically indicated an apparent and significant difference.

From the above, it has been proved that the apple juice enhances the anticancer effect and the cancer-suppressing effect.

In addition, the number of mice in which tumor onset was suppressed was 2 in the control group, and 6 in the group administered with apple juice. This indicates that tumor, once established, was removed by the elevated immune system of mice.

Further, it was observed in the group administered with apple juice that the initially established tumor grew to a size of 5.1 mm², and then disappeared.

In addition, mice showing a remarkable delayed the growth of the tumor were also observed in the group administered with apple juice.

On the other hand, the tumor of the mice in the control group administered with water alone continued to grow until it caused death of the subject mice.

However, it was observed in the group administered with apple juice that the size of the tumor was reduced as well as stopping the growth of the tumor.

This is considered to be due to the suppression of tumor growth and the necrosis of tumor cells by oral administration of the apple juice.

From the results of the repeated tests which were newly carried out, it has been observed that the apple juice further showed a remarkable antitumor effect.

The survival rate was 30% for the mice in the control group administered with water alone, but 80% for the mice in the group administered with apple juice, which was 50% greater than that of the mice in the Cordyceps sinensis group. Further, the life time of the mice where the tumor advanced to death was 40 days for the control group, 46.4 days for the Cordyceps sinensis group, but 77 days for the apple group, which indicates that the apple juice had about twice the life-prolonging effect.

In addition, the tumor size on the 28^{th} day after tumor inoculation, was 11.0 mm² (square sum of long diameter×short diameter) for the control group, 7.5 mm² for the Cordyceps sinensis group, but 1.3 mm² for the apple group, which indicated a remarkable suppression of the tumor growth.

These effects are better than those of anticancer hemolytic streptococcus formulation (OK-432; Trademark Picibanil) or an anticancer polysaccharide product (PSK; Trademark Krestin) which has been conventionally used as an immunopotentiator.

In addition, 20 female 4-week-old BALB/c mice (CLEA Japan, Inc.) were divided into two test groups, i.e., a test group administered with apple juice (Malus domestica), and a control group administered with sterilized water only. The mice which had taken the fruit juice (2% by volume) for 3 months every day were inoculated with a carcinogenic substance, azoxymethane, by subcutaneous injection.

For 3 months after the inoculation, the apple juice was administered every day. The mice were then dissected to measure the number and the size of the tumor in the esophagus, the stomach, and the intestine, to investigate the influence of the continuous administration of the apple juice on the action of the carcinogenic substance.

As the results, the number of the tumor was 10.6 for the control group, and 6.9 for the apple juice group on average, which indicates that the tumor was suppressed by about 65%, but no statistically significant difference was obtained.

However, the mean value of the surface area having an approximate oval shape was obtained by measuring the long diameters and the short diameters of tumors, which was 162.0 for the control group, and 90.6 for the group administered with the apple juice, which indicates that the tumor has been observed to be suppressed by about 50%. This difference is a statistically significant difference by a 3.0% level.

In other words, it has been found that the growth of cancer cells could be suppressed even when inoculated with a potent carcinogenic substance such as azoxymethane by continuously administering the apple juice.

The anticancer and cancer-suppressing effect by oral administration of the apple juice is considered to be caused from the enhanced systemic immune system by the apple itself or the fruit juice thereof which is absorbed from lymphoid follicle of the digestive tract or epithelial cells of the intestinal mucous membrane. Especially, it has been proven that phagocytosis activity of macrophages is remarkably enhanced.

In the following, the phagocytosis activity of macrophage which was discovered by the present inventors, will be explained.

In the above test showing the antitumor effect of the apple juice, 3 mice per test group in which the growth of inoculated MethA was suppressed to cure cancer, were investigated for the phagocytosis of a peritoneal macrophage.

The method used was a conventional method, wherein physiological saline was injected into the peritoneal cavity and the recovered macrophages were put into a MEM medium in a petri dish, and was incubated for 30 minutes in a CO₂ incubator.

Then, yeasts were given as a food and incubated for 1 hour. Then, May-Grunwald-Giemsa's stain was carried out, measuring the phagocytosis ability from the number of the yeasts phagocytosized by the macrophage.

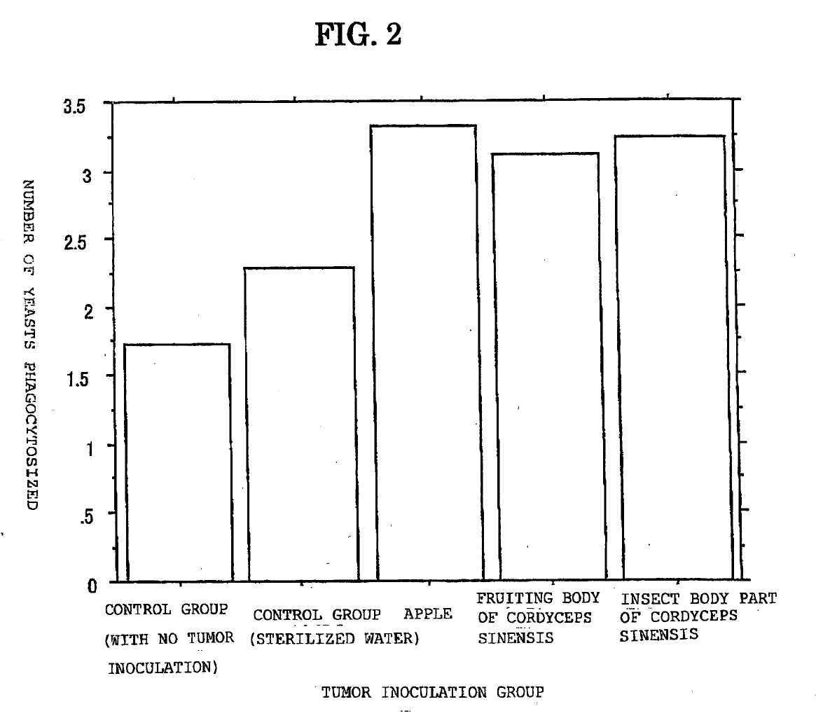

As the results shown in FIG. 2, a macrophage from the mice in the group inoculated with tumor showed a higher phagocytosis as compared to that from the group not inoculated with tumor. Especially, the phagocytosis ability of the macrophage of the mice having taken an apple juice was higher than that of mice in the group administered with the extract of Cordyceps sinensis. It was about 1.5 times higher than that of the mice in the group administered with sterilized water only as the control group.

Similar tests were repeatedly carried out for other mice, and as the results, the number of the yeasts which were phagocytosized by the macrophage of the mice was 2.0 for the control group on average, 4.0 for the Cordyceps sinensis group, and 4.3 for the apple group which had the highest number. This difference was statistically significant. The macrophage in the apple group showed twice the phagocytosis ability, as compared to the control group.

From these results, it was suggested that by taking apple juice continuously, the phagocytosis ability of macrophage was elevated to attack the tumor cells, and therefore, to elevate the antitumor effect.

Further, from the research for lymphocyte in a peripheral blood using a FACS (flow cytometry analysis), it has been found that the frequency or the activity of killer T-cells or helper T-cells in peripheral blood is also elevated, as shown below. Apple juice is considered to enhance the innate immune system such as natural killer cells, macrophages and the like, to inhibit the growth of cancer cells, and to prevent or suppress cancer.

On the other hand, cells taking part in the immune system except phagocytes such as macrophages or natural killer cells include the above-described lymphocytes.

The lymphocytes include B-cells and T-cells, and can identify specifically the other cells. The T-cells include killer T-cells which selectively attack abnormal cells such as cancer cells, and helper T-cells which help the action of the killer T-cells.

Next, the frequencies of such killer T-cells and the helper T-cells were investigated.

In the test showing the antitumor effect of the apple juice, apple juice was continuously administered to the mice in which tumor had been inoculated and cured, and then the frequencies of the helper T-cells and the killer T-cells in mice were obtained after 5 months.

As the results, the frequency of the helper T-cells of mice was 6% for the control group administered with water alone, 30.2% for the Cordyceps sinensis group, and 31.8% for the apple group, which was 5 times greater than the control group. However, there was no statistical difference between the Cordyceps sinensis group and the apple group.

The frequency of the killer T-cells which attacks directly the tumor cells was 1.8% for the control group, 7.7% for the Cordyceps sinensis group, and 10.4% for the apple group, which indicates that the apple group has the highest killer T-cell frequency.

There were statistically significant differences by a 3% level between the apple group and the Cordyceps sinensis group, and by 0.01% of probability between the apple group and the control group. In other words, it is considered that by continuously administering the apple juice, thereby removing the inoculated tumor MethA, the mice increase the frequency of lymphocytes such as killer T-cells which attack directly the cancer cells and helper T-cells which help the activity of the killer T-cells, and thus elevate the phagocytosis ability of natural killer cells and macrophages.

The apple juice was divided into the following seven groups in order to identify the ingredients contained in the apple juice having the antitumor effect or the immunopotentiating effect.
1: Juice from which only polyphenol was removed (pectin, enzyme and unknown substances).
2; Juice obtained by heating (1) (120° C., 20 minutes) to inactivate the enzyme contained in the juice.
3; Juice from which pectin was removed (polyphenol, enzyme and unknown substances).
4; Juice obtained by heating (3) (120° C., 20 minutes) to inactivate the enzyme.
5; Juice from which both polyphenol and pectin were removed (enzyme and unknown substances).
6; Juice obtained by heating (5) (120° C., 20 minutes) to inactivate the enzyme contained in the juice (unknown substances).
7; A control group which is administered with sterilized water alone.

Seven test groups were prepared as above, and such substances were administered directly to the peritoneal macrophage of the mice in each of the groups, and their influences on the phagocytosis ability of the macrophage were investigated.

Further, the test method was a conventional method, wherein 10 μl of a sample was put into 1000 μl (micro-liter) of a MEM medium.

As the results, the phagocytosis ability of macrophage was the highest in the test group (3) to which non-heated polyphenol was administered, and it was observed that there was a statistically significant difference.

On the other hand, the same 7 kinds of the substances were orally administered to mice (10 per test group) for 3 months, and then each of the peritoneal phagocytosis ability of macrophages in the mice was investigated. As the results, the highest value was observed in the group (1) to which non-heated pectin was administered, and it was also observed that there was a statistically significant difference, as compared to the other groups.

In other words, there was discrepancy between the test results when the ingredients of the apple juice were directly administered to macrophage, and the test results when the ingredients were orally administered to the mice and the phagocytosis ability was investigated after the peritoneal macrophage was taken out of the mice. However, it has been observed that both of cases are poorer in the phagocytosis ability, as compared to the macrophage of the mice administered with the apple juice itself, and therefore, administration of the apple juice itself can elevate the antitumor effect or the immunopotentiating effect more effectively than the separate administration of the ingredients of the apple juice.

In the modern pharmaceutical science or the food science, it is a standard procedure to analyze various substances contained in apple juice and the like, determine the molecular structures thereof, and these are separately administered for the purpose of investigating their respective antitumor effects or immunopotentiating effects.

However, it has been proven in this invention that such analytic method itself has some problems. In other words, it is considered that various substances act collectively to elevate the antitumor effect or the immunopotentiating effect of an apple juice.

It has been proven by the present inventors that apple juice as is, without separating the ingredients of the apple juice into the polyphenol or the pectin, can elevate the antitumor effect or the immunopotentiating effect of the fruit.

On the basis of the test results from the mice, studies on humans were carried out.

In other words, it was tested to determine if the apple juice as is, without heat-treatment or antiseptic treatment by vitamin C and the like, has a better immunopotentiating effect, as compared to Cordyceps sinensis.

Specifically, it was tested to determine if the activity of natural killer (NK) cells would be enhanced in the case where an apple with the fruit skin included and the fruit core removed, was made into a juice by a juicer, and was taken every day.

A research was planned based on Helsinki Declaration, wherein 37 volunteers (19 male, 18 female) took 2 apples (Fuji Apple) in the form of a juice every day for 5 weeks. Blood sample was taken before drinking, 2 weeks after the initiation of drinking, 5 weeks after initiation of drinking, and 5 weeks after termination of drinking to investigate an NK activity value, a blood glucose value, a neutral lipid value, a total cholesterol value, and a HDL-C (high cholesterol) value for each of the cases.

As the results, NK value before drinking was 38.8, 2 weeks after initiation of drinking 30.2, 5 weeks after initiation of drinking 46.8, and 5 weeks after termination of drinking 31.7, respectively.

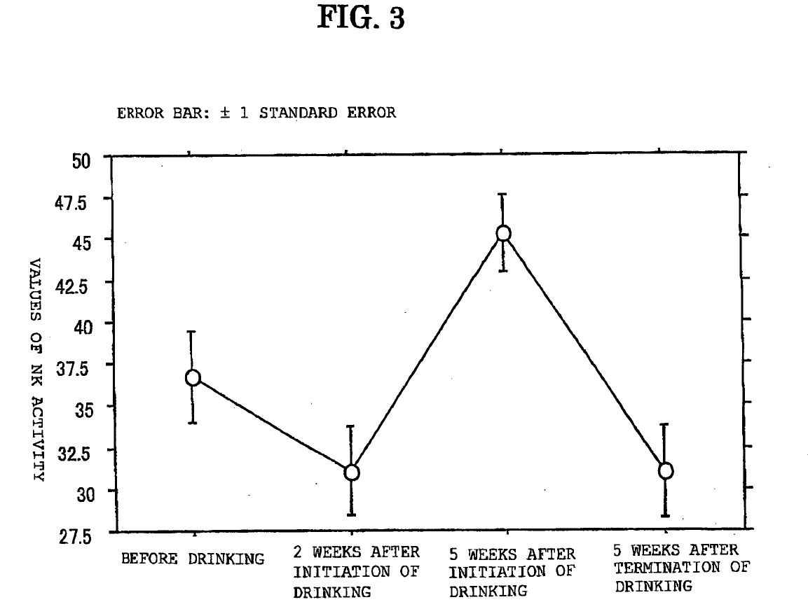

In other words, it has been observed that if the apple juice was continuously administered for 5 weeks, the NK activity value was enhanced in 5 weeks, which clearly indicated a statistically significant difference, as shown in FIG. 3.

Further, this value was greater than the results obtained when a yogurt containing Lactobacillus casei Shirota strain was taken for 3 weeks, which was carried out by Nakao (2000), et al.

In addition, it has been observed that with the lower NK activity value of the subject before drinking the apple juice, the higher the NK value was obtained, and further an abnormally low NK value of the subject was also returned to normal value.

Each of the blood glucose value, the neutral lipid value, the total cholesterol value and the HDL-C (high cholesterol) value before drinking apple juice was compared to each of those values 5 weeks after drinking the apple juice. As the results, it has been observed that the blood glucose value was significantly lowered.

By the Examples as above, it has been proven that apple juice has prominent anticancer and cancer-suppressing effects. Further, it has been observed that the system activates the “innate immune system” such as natural killer cells, macrophages and the like, as well as increase the frequency and the function of killer T-cells and helper T-cells.

In addition, these effects of an apple have been proven through the researches for humans, as well as that for mice.

On the other hand, it has been reported by Tazawa, et al., at Toyama Medical and Pharmaceutical University, that if the pectin from apple and the pectin from mandarin orange are administered to the rats in two groups, and then with a carcinogenic substance, azoxymethane, the group administered with the apple pectin showed a higher cancer-suppressing effect.

Further, it has been reported by Eberhardt, et al., at Cornell University (USA) that polyphenol of apple extracted by acetone has a much higher anti-oxidation activity as compared to Vitamin C.

The simply called “apple polyphenol” has more than 300 kinds of the substances such as flavonoid or katekin, thus it is still unclear which of them is the substance having the high anti-oxidation activity.

In the following, specific examples of apple juice which can be developed as a health food based on the present invention, will be described.

Almost the half of the immature fruits at the time of one or two month(s) after fruiting are thinned out and discarded for harvesting big fruits in the process of the so-called “fruit thinning.” Such immature fruits have polyphenol about 10 times more per unit weight, as compared to mature fruits.

It was tested to determine if the antitumor effect would be enhanced in the case where such an immature fruit with the fruit skin included and the fruit core removed, as a raw material, was made into a juice by a juicer and was administered to mice. Further, the antitumor effect when the juice of such immature fruit is mixed with a juice of a mature fruit, was also investigated.

In addition, the antitumor effect and the immunopotentiating effect of crabapple (Malus pumila) or a fruit of the genus Pyrus which has more polyphenol per unit weight, as compared to apple Malus domestica, were investigated in the same manner as in the above-described test. The fruit was made into a juice with the fruit skin included and the fruit core removed by a juicer and taken by mice. Further, the antitumor effect was investigated when crabapple (Malus domestica) was made into a juice by a juicer with the fruit skin included, and mixed with a juice of a mature fruit of the genus Malus domestica. This was carried out for the purpose of developing a functional food or a drug which elevates the antitumor effect and the immunopotentiating effect by enhancing the frequencies of pectin and polyphenol by mixing the pectin which is contained in a large amount in the juice of a mature fruit of the genus Malus domestica, with the polyphenol which is contained in a large amount in a fruit of the genus Malus pumila (crabapple) or a fruit of the genus Pyrus.

As the results of the test for the mice, it has been observed that a juice of a mature fruit of the genus Malus domestica which is mixed with a juice of an immature fruit thereof, a fruit juice of crabapple (Malus domestica), or with the genus Pyrus, when administered, has a higher antitumor effect or immunopotentiating effect, as compared to a juice of a mature fruit of the genus Malus domestica alone. Further, it has been proven that such mechanism activates the “innate immune system” such as natural killer cells, macrophages and the like, as well as increases the frequency and the function of killer T-cells or helper T-cells.

In addition, a dog (Siberian husky) born on Jan. 27, 1989 was found to have a tumor having a size as of a quail's egg or more at the right lower thigh on Mar. 19, 2001 (at age 13), which was removed by surgical operation. However, it was found that the tumor spread to the lung (1.5 to 2.0 cm) on May 21, 2002. The remaining life expectancy was 1 to 2 month(s), as diagnosed. To the dog which could not even drink water, was orally administered with apple juice made by a juicer using an apple with the fruit skin included and the fruit core removed in early June 2002. An apple (Fuji Apple) made into a juice by a juicer was continuously administered in the mornings and nights two times every day. As the result of examination on Oct. 5, 2002, no tumor was found.

Further, a dog (Siberian husky) born on Mar. 22, 1992 was found to have a tumor having a size of the end of a little finger at the very end of the left breast on May 21, 2002 (at age 10). An apple (Fuji Apple) made to a juice by a juicer using an apple with the fruit skin included and the fruit core removed was continuously administered in the mornings and nights two times every day. As the result of examination on Oct. 5, 2002, no tumor was found.

Apple or the juice thereof which has been proven to have the anticancer effect in the present invention, has been provided as a food for a long time, thus it is already known that it has no side effect.

As described above, apple or the juice thereof which has a high immunopotentiating effect, can be used as an anticancer agent, as well as a health food product or a health drink product.

As described above, apple or the juice thereof, when taken regularly, can prevent cancer.

Thus, the present invention can save humans from cancer, enhance QOL (Quality Of Life) of cancer patients, significantly contributing to medical care of humans or animals, as well as to the development of the agricultural industry or the apple industry, the related process industries and the like.

In the above Examples, juice of a fruit of the genus Malus such as apple Malus domestica or crabapple Malus pumila was orally administered as a fruit of the genus Malus. However, the same effects as in the above Examples were obtained by the tests wherein other substance, i.e., a fruit of the genus Malus such as Malus sieboldii, a fruit of the genus Pyrus such as pear, a substance derived therefrom, or an aqueous extract of the fruit of the genus Malus or a substance derived from the aqueous extract, an extract of the fruit of the genus Malus obtained by extraction with an organic solvent such as alcohol, or a substance derived from the extract, was used and administered percutaneously, parenterally or via the routes other than an oral route.

In addition, the same effects as in the above Examples were obtained by the tests wherein, the above-described fruit such as an apple or a pear was administered in a mixture with, for example, a fruit of the genus Prunus such as an apricot, a plum, a peach or a mountain cherry, a fruit of the genus Chaenomeles such as a Flowering quince, a fruit of the genus Cydonia such as a marmelo, a fruit of the genus Diospyros such as a persimmon, a fruit of the genus Morus such as a mulberry, a fruit of the genus Rosa such as a Japanese rose, a fruit of the genus Poncrirus such as a trifoliate orange, or a fruit of the genus Eriobotrya such as a loquat, or wherein, for example, a fruit of the genus Prunus such as an apricot, a plum, a yellow peach or a mountain cherry, a fruit of the genus Chaenomeles such as a Flowering quince, a fruit of the genus Cydonia such as a marmelo, a fruit of the genus Diospyros such as a persimmon, a fruit of the genus Morus such as a mulberry, a fruit of the genus Rosa such as a Japanese rose, a fruit of the genus Poncrirus such as a trifoliate orange, or a fruit of the genus Eriobotrya such as a loquat, was administered alone.

### Industrial Applicability

As described above, it has been observed that the invention described in claims 1 to 3 or 6 in the present inventions has the effects that a fruit of the genus Malus such as apple (Malus domestica) and crabapple (Malus pumila), a fruit of the genus Pyrus, a substance derived therefrom, an extract by extracting the fruit with water or an organic solvent such as alcohol, or a substance derived from the extract, when administered, enhances the innate immune system such as natural killer cells, macrophages and the like to control cancer-attacked cells, as well as to prevent carcinogenesis of cells not yet attacked by cancer, and when taken regularly, has the effect of preventing the onset of cancer, that is, the cancer preventing effect.

In other words, administration of a fruit of the genus Malus such as apple (Malus domestica) and crabapple (Malus pumila), a fruit of the genus Pyrus, or a substance derived therefrom could enhance the innate immune system such as natural killer cells, macrophages and the like, and the activity of lymphocytes, inhibit the onset or growth of cancer cells, and prevent or suppress cancer.

Further, it has been proven that it could suppress or control cancer more effectively, as compared to the extract of Cordyceps sinensis which has conventionally been suggested as an immunopotentiator.

Further, it has been found that by administering a fruit of the genus Malus such as apple (Malus domestica) and crabapple (Malus pumila), a fruit of the genus Pyrus, or a substance derived therefrom to cancer patients, QOL of the cancer patients is improved, and remarkable life-prolonging effect is obtained.

Accordingly, it can be widely administered since it is not expensive and is more easily available, as compared to the conventional extract of Cordyceps sinensis.

The invention described in claim 4 has the effect of enabling suitable administration depending on the situation percutaneously, parenterally or via other routes, although oral administration is a general administration route, in addition to the effects of the invention described in claims 1 to 3.

It has been proven that the invention described in claims 5 and 7 has the effect that a mixture of a mature fruit of the genus Malus domestica (apple) with an immature fruit thereof or a fruit of crabapple (Malus domestica) has a higher antitumor effect or a higher immunopotentiating effect, as compared to a mature fruit of the genus Malus domestica alone, in addition to the effects of the invention described in claims 1 to 4 or 6.

Further, in the case of using an immature fruit, an immature fruit which has conventionally been thinned out and discarded can be harvested and utilized as a juice. In addition, it can be harvested without spreading insecticides or antiseptics since it is harvested before being infected by harmful insects or diseases.

Therefore, an organic fruit juice can be easily prepared, in addition to saving cost for agricultural chemicals, which contributes greatly to the development of industrial applications as a new raw material for a health food

1. A drug such as an immunopotentiator, comprising both a mature fruit and an immature fruit of a fruit of the genus Malus such as apple (Malus domestica) and crabapple (Malus pumila), a fruit of the genus Pyrus, or a substance derived therefrom as an active ingredient.

2. The drug such as an immunopotentiator as described in claim 1, comprising an aqueous extract of both a mature fruit and an immature fruit of the fruit of the genus Malus such as apple (Malus domestica) and crabapple (Malus pumila), the fruit of the genus Pyrus, or a substance derived from the aqueous extract.

3. The drug such as an immunopotentiator as described in claim 1, comprising an extract of both a mature fruit and an immature fruit of the fruit of the genus Malus such as apple (Malus domestica) and crabapple (Malus pumila), the fruit of the genus Pyrus obtained by extraction with an organic solvent such as alcohol, or a substance derived from the extract.

4. The drug such as an immunopotentiator as described in claim 1 administered orally, percutaneously, parenterally or via other routes, which comprises the extract of both the mature fruit and the immature fruit of the fruit of the genus Malus such as apple (Malus domestica) and crabapple (Malus pumila), the fruit of the genus Pyrus, or a substance derived from the extract as a raw material.

5. (canceled)

6. A health food for enhancing immune system, comprising both a mature fruit and an immature fruit of a fruit of the genus Malus such as apple (Malus domestica) and crabapple (Malus pumila), a fruit of the genus Pyrus, or a substance derived therefrom as a raw material.

7. (canceled)

8. The drug such as an immunopotentiator as described in claim 2 administered orally, percutaneously, parenterally or via other routes, which comprises the extract of both the mature fruit and the immature fruit of the fruit of the genus Malus such as apple (Malus domestica) and crabapple (Malus pumila), the fruit of the genus Pyrus, or a substance derived from the extract as a raw material.

9. The drug such as an immunopotentiator as described in claim 3 administered orally, percutaneously, parenterally or via other routes, which comprises the extract of both the mature fruit and the immature fruit of the fruit of the genus Malus such as apple (Malus domestica) and crabapple (Malus pumila),
